(19) United States
(12) Patent Application Publication
(10) Pub. No.: US 2023/0355445 A1
(43) Pub. Date: Nov. 9, 2023

(54) DIAPER SYSTEM COMPRISING AN EXTERNAL ENVELOPE, AN ABSORBENT ELEMENT AND POUCH

(71) Applicant: Generation Plume, Paris (FR)

(72) Inventor: Florence Hallouin, Paris (FR)

(21) Appl. No.: 18/246,069

(22) PCT Filed: Sep. 21, 2021
(86) PCT No.: PCT/FR2021/051616

§ 371 Date: Mar. 21, 2023

(30) Foreign Application Priority Data
Sep. 21, 2020 (FR) FR2009564

(51) Int. Cl. A61F 13/15

(52) U.S. Cl. CPC A61F 13/15585; A61F 2013/15878; A61F 2013/15918

(57) ABSTRACT

A diaper system comprises an outer envelope, an absorbent element and a pouch having an opening for partially inserting the absorbent element and means for fastening onto the outer envelope. The pouch forms, before use, a flat sheath, and comprises a superposition of two sheets of a flexible and leaktight material. At least one of the surfaces of the two sheet is heat-activatable. One of the sheets has a cutout corresponding to the opening, and the two sheets are assembled by two transverse lines and two longitudinal lines in the peripheral edge strip of the sheets.

### CROSS-REFERENCE TO RELATED APPLICATIONS

This application is a national phase entry under 35 U.S.C. § 371 of International Patent Application PCT/FR2021/051616, filed Sep. 21, 2021, designating the United States of America and published as International Patent Publication WO 2022/058699 A1 on Mar. 24, 2022, which claims the benefit under Article 8 of the Patent Cooperation Treaty to French Patent Application Serial No. FR2009564, filed Sep. 21, 2020.

### TECHNICAL FIELD

The present disclosure relates to the field of absorbent diapers intended to be worn around the waist for preventing the leakage of body secretions at the edges, around the crotch region.

The present disclosure relates more particularly to absorbent diapers for babies and for incontinent people, which are economically reusable and environmentally friendly, and are simultaneously comfortable, impermeable, easy to use, and partially reusable.

It also relates to feminine hygiene applications.

### BACKGROUND

European patent EP2389151 is known in the art, and describes a reusable absorbent diaper, consisting of:
an outer envelope, generally made of fabric, and preferably of fabric with elastic fibers, for example, elastane, a polyurethane derivative. This outer envelope is reusable and washable.
an intermediate element, generally made of an impermeable textile or a textile made impermeable by a polyurethane coating, such as GORTEX®. This intermediate element is also, preferably, reusable and washable. The fabric of the intermediate element has a characteristic total weight (fabric+coating) of about 105 g/m² with ±30 g/m².
an absorbent element, either disposable, for example, a lining composed mainly of cellulose, or washable and reusable, made of an absorbent fabric such as hemp, cotton, bamboo, and/or synthetic fiber.

The intermediate element of this solution from the prior art forms a sort of pocket or pouch, fixed at each transverse end to the outer element. This intermediate element contains the absorbent element and ensures the sealing.

The main element, from a functional point of view, of the aforementioned prior art diapers is the intermediate element which must provide a plurality of functions of attaching the two other elements, inner and outer, as well as sealing and ergonomics, in a material having mechanical and chemical properties.

The solution proposed in the prior art consists in making a pocket with a gathered opening. The production of such a configuration is possible by a craft process, which is poorly suited to industrial production, at high production rates and a moderate manufacturing cost.

### BRIEF SUMMARY

In order to remedy these drawbacks, the present disclosure relates in its most general sense to a diaper system comprising an outer envelope, an absorbent element and a pouch having an opening for inserting the absorbent element and means for fastening onto the outer envelope, the pouch forming, before use, a flat sheath, characterized in that the pouch comprises a superposition of two sheets of a flexible and leaktight material, at least one of the surfaces of which is heat-activatable, one of the sheets having a cutout corresponding to the opening, the two sheets being assembled by two transverse lines and two longitudinal lines in the peripheral edge strip of the sheets.

Preferably, the two sheets each have a heat-fusible surface, the sheets being superimposed so that the heat-fusible surfaces are in contact, and are assembled by heat-welding of transverse and longitudinal lines in the peripheral edge of the sheets.

The present disclosure also relates to a pouch having an opening for the partial insertion of an absorbent element and means for fastening onto an outer envelope, characterized in that it comprises a superposition of two sheets, in particular, rectangular or trapezoidal, of a flexible and leaktight material, one of the surfaces of which is heat-activatable, one of the sheets having a cutout corresponding to the opening, the two sheets being assembled by welding the adjacent heat-activatable surfaces along peripheral lines and/or along two transverse lines and two longitudinal lines in the peripheral edge strip of the sheets.

The present disclosure also relates to a method for manufacturing such a pouch, characterized in that it comprises the following steps:
Preparing two stacks of sheets of a flexible material, one surface of which is heat-activatable, the sheets of one of the stacks having a cutout forming the opening;
Depositing a first sheet from one of the stacks on a second sheet from the other of the stacks, the sheets being placed with heat-activatable surface against heat-activatable surface; and
Bonding, by heat-welding or ultrasonic welding or gluing along transverse and longitudinal lines in the peripheral edge of the superimposed sheets.

According to one variant, the method comprises the following steps:
Superimposing a first upper sheet on a second lower sheet, the sheets being formed of a flexible material, one surface of which is heat-activatable, one of the sheets having a cutout forming the opening, the sheets being placed with heat-activatable surface against heat-activatable surface; and
Heat-welding or any other gluing or welding method along transverse and longitudinal lines in the peripheral edge of the superimposed sheets.

According to a first variant, the method comprises a prior step of preparing two stacks of sheets of a flexible material, one surface of which is heat-activatable, the sheets of one of the stacks having a cutout forming the opening.

According to a second variant, the upper sheets come from a roll, the method comprising an additional step of transverse cutting after heat-welding with a lower sheet.

Preferably, the upper sheets come from a roll of a flexible material, one surface of which is heat-activatable, the method comprising an additional step of transverse cutting after heat-welding with a lower sheet.

According to a particular embodiment, the lower sheets come from a roll of flexible film, one surface of which is heat-activatable, supplying a conveyor belt, the method comprising an additional step of transverse cutting after heat-welding with a sheet with lower.

According to one variant, the method comprises a step of cutting the opening in the unrolled film on the conveyor belt before the heat-welding step.

The present disclosure also relates to an apparatus for manufacturing an aforementioned pouch, characterized in that it comprises a station for assembling a first upper sheet on a second lower sheet, the sheets being formed of a flexible material, one surface of which is heat-activatable, one of the sheets having a cutout forming the opening, the sheets being placed with heat-activatable surface against heat-activatable surface, and a tool for heat-welding along transverse and longitudinal lines in the peripheral edge of the superimposed sheets.

### BRIEF DESCRIPTION OF THE DRAWINGS

The present disclosure will be better understood on reading the following description, with reference to the appended drawings illustrating non-limiting embodiments in which:

FIG. 1 shows a schematic view of an example of a diaper system according to the present disclosure;

FIG. 2 shows an example pouch according to the present disclosure; and

FIG. 3 shows a schematic view of an embodiment of an apparatus according to the present disclosure.

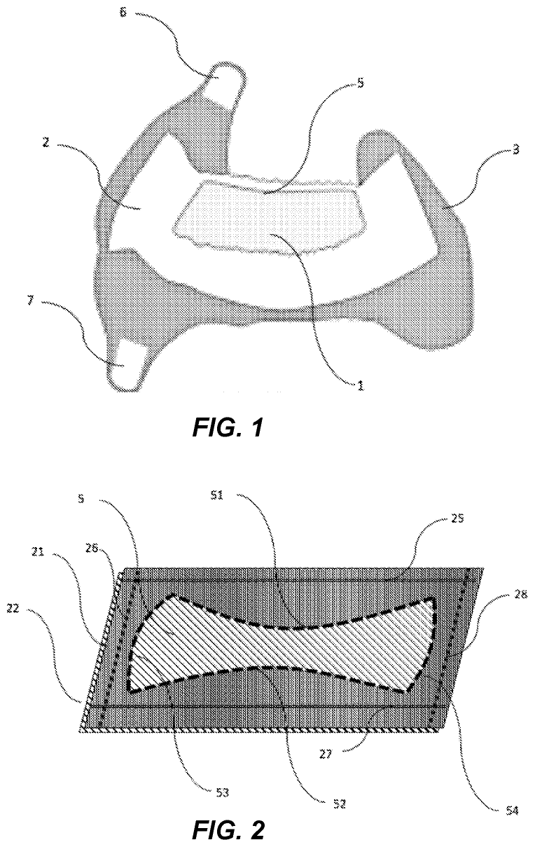

### DETAILED DESCRIPTION

General Principle of the Diaper System

The general principle of the diaper system is that described in patent EP2389151. It comprises an absorbent element (1) positioned in a pouch (2) ensuring a leaktight hold on the body of the user, the pouch (2) being reversibly fixed to an outer envelope (3).

The absorbent element (1) may preferably be washable, in the form of a foldable, microfiber or organic cotton towel, or in disposable or compostable or biodegradable form. It is inserted into the pouch (2), which forms a pocket opening through an opening having a smaller cross-section than that of the absorbent element (1).

The outer envelope (3) can be adapted to the wearer's body by fasteners, press buttons, gripping elements (6, 7), one or more elastics, or others, arranged at the wearer's waist. The outer envelope (3) may be underwear, i.e., a part that fits directly on the waist of the wearer and comprises an opening for each of their thighs.

Details of the Pouch (2)

FIG. 2 shows a detailed perspective view of the pouch (2). It is formed by assembling two sheets (21, 22) made of a flexible material, one of the surfaces of which is heat-activatable. The upper sheet (21) has an opening (5) delimited by two concave longitudinal edges (51, 52) and two concave transverse edges (53, 54).

The length of the opening (5), measured on the longitudinal axis, is between 65% and 80% of the length of the sheath, the width, measured on the transverse axis of the transverse edges, is between 30% and 50% of the width of the pouch (2). The edges of the opening (5) can be gathered.

The pouch can be extended on either side of its length by stiffeners ensuring the connection with the outer envelope (3), for example, flexible plastic blades slid into a hem provided at the two transverse ends of the pouch (2). These hems can be formed by second transverse heat-welding lines, parallel to the lines (26, 28). The pouch (2) can also be extended on either side of its length by seams, grippers, press buttons or any other attachment system connecting the pouch (2) to the outer envelope (3).

Sheet Shape

The two sheets (21, 22) are, in one embodiment, simple rectangular shapes of the same dimensions.

In order to adapt the pouch more ergonomically and anatomically, they can also have a trapezoidal shape, in order to offer a larger width on one side, which will correspond to the buttocks.

According to another configuration, the longitudinal edges are not rectilinear but convex, the sheets then having a barrel shape. In this case, it is preferable to provide a rectangular cutout and not with concave longitudinal edges. This shape makes it possible to increase the depth of the pouch (2) to receive the absorbent element (1).

The two sheets (21, 22) may also have different dimensions, in particular, for the width. The upper sheet (21) having the cutout is, in this case, slightly smaller than the lower, solid sheet (22), the edge of the latter receiving an elastic in order to improve the tightening and the sealing. This edge may also be gathered or cut in the manner of lace for better aesthetics.

Variants Relating to the Welding and Edge Lines

The two sheets can be welded with a continuous thread or with transverse segments and longitudinal segments crossing in the corners. The border may be thin, such as a few millimeters, or wider, such as a few centimeters. In this case, the weld line leaves two peripheral lips on the outside between which it is possible to deposit a longitudinal or peripheral elastic strip or a transverse stiffener, held by gluing or welding. It is also possible to then close these two lips by an additional weld line in order to form a hem enclosing the elastic or the stiffener.

The weld lines are not necessarily parallel to the edges of the sheets (21, 22); they can be convex in order to increase the volume of the pouch, or curved to form a wasp waist in order to improve the anatomical fit of the absorbent, which is itself anatomical.

Production Apparatus

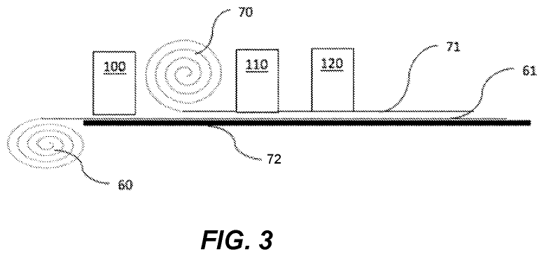

FIG. 3 shows an example of an apparatus, comprising a first unwinder (60) delivering a film (61) of flexible material, the upper surface of which is heat-activatable, driven by a suction conveyor belt (72).

A first workstation (100) comprises a cutting die cutting the opening (5) in the film.

A second unwinder (70) then deposits on the film (61) a second film (71) of flexible material in the heat-activatable surface is oriented toward the first film (61). A heat-welding station (110) provides the connection along two longitudinal heat-welding lines and two transverse heat-welding lines. A cooling station for the tool can be added to this heat-welding station in order to keep the welding tools at a constant temperature.

A third station (120) then ensures the cutting of the opening by a transverse cut.

In addition, the apparatus further comprises a station for depositing a transverse semi-rigid insert, and an additional transverse welding station for closing the hem after placing the insert.

Compostable Version

The materials constituting the absorbent element, and optionally those forming the pouch, are advantageously made of natural materials, for example, cotton and linen, or cellulose or any other biodegradable or compostable materials and, for the pouch, this is sealed with starch or any other biodegradable or compostable plastic, to allow composting.

1. A diaper system, comprising:
an outer envelope;
an absorbent element; and
a pouch having an opening for partially inserting the absorbent element into the pouch and means for fastening the pouch onto the outer envelope, the pouch forming, before use, a flat sheath, wherein the pouch comprises a superposition of two sheets of a flexible and leaktight material, at least one of the surfaces of which is heat-activatable, one of the sheets having a cutout corresponding to the opening, the two sheets being assembled by two transverse lines and two longitudinal lines in the peripheral edge strip of the sheets.

2. The diaper system of claim 1, wherein each of the two sheets has a heat-fusible surface, the sheets being superimposed so that the heat-fusible surfaces are in contact, and are assembled by heat-welding of transverse and longitudinal lines in the peripheral edge of the sheets.

3. A pouch having an opening for the partial insertion of an absorbent element into the pouch and means for fastening of the pouch onto an outer envelope, wherein the pouch comprises a superposition of two sheets of a flexible and leaktight material, one of the surfaces of the two sheets being heat-activatable, one of the two sheets having a cutout corresponding to the opening, the two sheets being assembled by two transverse lines and two longitudinal lines in a peripheral edge strip of the two sheets.

4. The pouch of claim 4, wherein each of the two sheets has a heat-fusible surface, the two sheets being assembled by welding the adjacent heat-fusible surfaces along peripheral lines.

5. A method for manufacturing a pouch according to claim 3, wherein the method comprises the following steps:
preparing two stacks of sheets of a flexible material, one surface of which is heat-activatable, the sheets of one of the stacks having a cutout forming the opening;
depositing a first sheet from one of the stacks on a second sheet coming from the other of the stacks, the first sheet and the second sheet being placed with heat-activatable surface against heat-activatable surface; and
heat-welding along transverse and longitudinal lines in the peripheral edge of the first sheet and the second sheet.

6. A method for manufacturing a pouch according to claim 3, wherein the method comprises the following steps:
superimposing a first upper sheet on a second lower sheet, the sheets formed of a flexible material, one surface of the sheets being heat-activatable, one of the sheets having a cutout forming the opening, the sheets being placed with heat-activatable surface against heat-activatable surface; and
heat-welding along transverse and longitudinal lines in the peripheral edge of the superimposed first sheet and second sheet.

7. The method of claim 6, further comprising a prior step of preparing two stacks of sheets of a flexible material, at least one surface of which is heat-activatable, the sheets of one of seethe stacks having a cutout forming the opening.

8. The method of claim 6, wherein the upper sheets come from a roll, and the method further comprises an additional step of transverse cutting after heat-welding with a lower sheet.

9. The method of claim 7, wherein the upper sheets come from a flexible roll, one surface of which is heat-activatable, and the method further comprises an additional step of transverse cutting after heat-welding with a lower sheet.

10. The method of claim 6, wherein the lower sheets come from a roll of flexible film, one surface of which is heat-activatable, supplying a conveyor belt, and the method further comprises an additional step of transverse cutting after heat-welding with a lower sheet.

11. The method of claim 10, further comprising a step of cutting the opening in the unrolled film on the conveyor belt before the heat-welding step.

12. An apparatus for manufacturing a pouch according to claim 3, the apparatus comprising:
a station for assembling and superimposing a first upper sheet on a lower second sheet, the sheets being formed of a flexible material, one surface of which is heat-activatable,
a tool for heat-welding along transverse and longitudinal lines in a peripheral edge of the superimposed sheets in order to weld the assembled sheets;
a first unwinder delivering a first film of flexible material, the upper surface of which is heat-activatable, driven by a suction conveyor belt;
a first workstation comprising a cutting die configured to cut an opening in a film, and
a second unwinder-E for depositing on the film a second film of flexible material, the heat-activatable surface of which is oriented toward the first film.
